(19) United States
(12) Patent Application Publication
(10) Pub. No.: US 2009/0246281 A1
(43) Pub. Date: Oct. 1, 2009

(54) Soft steroid compositions for use in dry powder inhalers

(75) Inventors: Michael I. Goller, London (GB); Qi Li, Pinecrest, FL (US); Jade C.Y. Ly, Cooper City, FL (US); Mohammed Nurul Momin, London (GB); Katherine Salas, Sunny Isles Beach, FL (US); Anayo Michael Ukeje, London (GB); Ramesh Yanamandra, London (GB); Xian-Ming Zeng, Surrey (GB)

Correspondence Address: KENYON & KENYON LLP, ONE BROADWAY, NEW YORK, NY, 10004, US

(73) Assignee: Norton Healthcare Ltd., London (GB)

(21) Appl. No.: 10/578,233

(22) PCT Filed: Nov. 3, 2004
(86) PCT No.: PCT/US04/36477

§ 371 Date: Jan. 30, 2009

Related U.S. Application Data
(60) Provisional application No. 60/517,324, filed on Nov. 3, 2003.

(51) Int. Cl. A61K 9/14; A61K 31/56; A61P 11/06

(52) U.S. Cl. 424/489; 514/179; 514/177

(57) ABSTRACT

A medicament and a method of producing a medicament are disclosed. The medicament contains a soft steroid and is suitable for administration via a dry powder inhaler.

### FIELD OF THE INVENTION

This invention relates to compositions containing soft steroids, and methods of preparation thereof. In particular, this invention provides soft steroid-based medicaments suitable for administration via a dry powder inhaler.

### BACKGROUND OF THE DISCLOSURE

Steroids operate by stimulating the transcription of certain genes, producing a variety of cellular effects, with different steroids activating different genes. Steroids are therefore capable of being used in a wide range of treatments. In particular, conventional corticosteroids, such as glucocorticoids, have been commonly used as anti-inflammatory agents for the treatment of inflammatory conditions and allergies.

Steroid treatments are often administered by the use of inhalation techniques, including the use of pressurised metered dose inhalers and dry powder inhalers. Such administration techniques have the advantage of transmitting the medicament through the alveoli directly into the blood stream as opposed to oral administration whereby the medicament must first pass through the digestive system. Administration via inhalation techniques provides an additional advantage in treating lung-based afflictions, such as cystic fibrosis, emphysema, bronchitis, asthma, etc., in that the medicament is delivered directly to the site of action within the respiratory tract. An example of this is the use of the corticosteroid budesonide as an anti-inflammatory agent, which agent is most commonly used in dry powder inhalers for the treatment of asthma.

Use of conventional steroid-based medicaments car, however, produce undesirable side effects which may severely limit their usefulness. Besides the consequences that result from the suppression of the hypothalamic-pituitary-adrenal (HPA) axis, prolonged therapy with corticosteroids is also limited by complications such as fluid and electrolyte abnormalities, hypertension, hyperglycemia, increased susceptibility to infection, osteoporosis, myopathy, behavioural disturbances, cataracts, growth arrest and fat distribution.

Soft drugs are a new therapeutic concept using agents that undergo predictable metabolism to inactive metabolites after exerting their therapeutic effect. Hence, they are designed by building into the molecule, in addition to the required activity, a desirable way in which the molecule is to be deactivated or detoxified. Ideally, the molecule is delivered to the site of action (e.g. the lung) and subsequently deactivated on absorption, thus reducing the likelihood of adverse events.

Recently, a new class of androstene-derived steroids, commonly known as soft steroids, have been developed which exhibit anti-inflammatory effects similar to those of conventional corticosteroids without the serious systemic side effects associated therewith Examples of soft steroids having anti-inflammatory activity are disclosed in U.S. Pat. No. 4,710,495, EP-B-0334853 and U.S. Pat. No. 5,981,517.

Pressurised metered dose inhalers, using volatile propellants usually containing some aqueous solvent such as ethanol, have tended to be used in preference over dry powder inhalers for administering conventional steroids in the treatment of asthma.

Although soft steroid substitutes for budesonide, such as etiprednol dicloacetate, are potentially useful as medicaments, such soft steroid-based medicaments which are administered via pressurised metered dose inhalers containing aqueous solvents have proved difficult to formulate, due to their instability in the traditional solvents. Moreover, soft steroid medicaments based on conventional dry powder formulations will demonstrate poor efficiency when used in dry powder inhalers for the treatment of asthma.

Consequently, it is an object of the present invention to provide a soft steroid-based formulation, which when formulated as a medicament for patient application via an inhalation technique, such as via a dry powder inhaler, can demonstrate improved stability characteristics and a high efficiency of delivering the drug to the site of action within the respiratory tract.

### SUMMARY OF THE INVENTION

The invention is directed towards a medicament suitable for use in a dry powder inhaler comprising particles of at least one soft steroid and at least one excipient, wherein said at least one soft steroid particles have an average volume mean diameter of less than about 20 micrometers and said at least one excipient particles have an average volume mean diameter in the range of about 10 to about 1000 micrometers.

The invention further envisages a method of preparing a medicament suitable for use in a dry powder inhaler, said method comprising admixing particles of a soft steroid and an excipient, wherein said steroid particles have an average volume mean diameter of less than about 20 micrometers and said excipient particles have an average mean diameter in the range of about 10 to about 1000 micrometers, as well as a method of treating a mammal with a medicament administered by way of a dry powder inhaler, said medicament comprising particles of a soft steroid and an excipient, wherein said steroid particles have an average volume mean diameter of less than about 20 micrometers and said excipient particles have an average mean diameter in the range of about 10 to about 1000 micrometers.

The invention also contemplates the use of a composition in the preparation of a medicament for the treatment of a mammal, said composition comprising particles of a soft steroid and an excipient, wherein said steroid particles have an average volume mean diameter of less than about 20 micrometers and said excipient particles have an average mean diameter in the range of about 10 to about 1000 micrometers.

The invention further includes a composition comprising particles of a soft steroid and an excipient, wherein said steroid particles have an average volume mean diameter of less than about 20 micrometers and said excipient particles have an average mean diameter in the range of about 10 to about 1000 micrometers.

It has been found that the efficacy of soft steroid based medicaments for use with dry powder inhalers can be improved by preparing medicaments in which both the soft steroid and an associated excipient are provided in a controlled range of particle sizes.

In one aspect, the present invention provides a medicament suitable for use in a dry powder inhaler comprising particles of a soft steroid and an excipient, wherein said steroid particles have an average volume mean diameter of less than about 20 micrometers and said excipient particles have an average mean diameter in the range of about 10 to about 1000 micrometers.

In another aspect, the present invention provides a method of preparing a medicament suitable for use in a dry powder inhaler, said method comprising admixing particles of a soft steroid and an excipient, wherein said steroid particles have an average volume mean diameter of less than about 20 micrometers and said excipient particles have an average mean diameter in the range of about 10 to about 1000 micrometers.

In another aspect, the present invention provides a method of treating a mammal with a medicament administered by way of a dry powder inhaler, said medicament comprising particles of a soft steroid and an excipient, wherein said steroid particles have an average volume mean diameter of less than about 20 micrometers and said excipient particles have an average mean diameter in the range of about 10 to about 1000 micrometers.

In another aspect, the present invention provides a composition comprising particles of a soft steroid and an excipient, wherein said steroid particles have an average volume mean diameter of less than about 20 micrometers and said excipient particles have an average mean diameter in the range of about 10 to about 1000 micrometers.

In another aspect, the present invention provides the use of a composition in the preparation of a medicament for the treatment of a mammal, said composition comprising particles of a soft steroid and an excipient, wherein said steroid particles have an average volume mean diameter of less than about 20 micrometers and said excipient particles have an average mean diameter in the range of about 10 to about 1000 micrometers.

In another aspect, the present invention provides the use of a composition in the preparation of a medicament for the treatment of a respiratory tract or lung disease or disorder in a mammal, said composition comprising particles of a soft steroid and an excipient, wherein said steroid particles have an average volume mean diameter of less than about 20 micrometers and said excipient particles have an average mean diameter in the range of about 10 to about 1000 micrometers.

Medicaments of the invention can be formulated for administration nasally or orally by way of dry powder inhalers, including both pre-metered inhalers such as those with blister and capsule cartridges, and by way of metered dose inhalers such as the AIRMAX (trade mark of the Ivax Corporation) inhaler.

The medicaments are preferably administered to humans, preferably for the treatment of respiratory tract or lung based diseases or disorders, such as asthma.

Without wishing to be limited by this possible explanation for the improved efficacy, it is believed the fine particle fraction (FPF), i.e. the percentage of drug that is likely to reach the lower airway under the normal conditions of use of the conventional formulations of the prior medicaments are too low for the soft steroid to exert satisfactory therapeutic effects. When inhaling the medicaments of the present invention, however, the soft steroid is passed readily into the lower lungs and thereby contacts many more of the potential contact sites in the lungs. By contacting the soft steroid with more sites in the lungs, the medicament of the present invention is capable of demonstrating an improved efficacy over the prior medicaments.

### BRIEF DESCRIPTION OF DRAWINGS

FIG. 1 is a scanning electron micrograph showing micronised etiprednoal dicloacetate (EDA) at ×2000 magnification.

FIG. 2 is a scanning electron micrograph showing a typical lactose batch at ×200 magnification.

FIG. 3 is a scanning electron micrograph showing a typical blend containing lactose and a soft steroid, etiprednol dicloacetate (EDA) at ×100 magnification.

FIG. 4 is the particle size distribution of several batches of etiprednol dicloacetate

### DESCRIPTION OF THE PREFERRED EMBODIMENTS

In order to provide a soft steroid medicament with improved efficacy when delivered by way of dry powder inhalers, both the soft steroid and its associated excipient should be provided within a controlled range of particle sizes. The volume mean diameter (VMD) of the soft steroid particles should, on average, be less than about 20 micrometers, whilst the VMD of the excipient particles should, on average, be in the range of about 10 to about 1000 micrometers. With medicaments prepared in accordance with these ranges, the soft steroid can be administered via inhalation; the soft steroid particles being transmitted deep into the lung by the inhaled air stream. Such formulations therefore enable delivery of the medicament via dry powder inhalers having excellent uniformity of dose per delivery with a high fine particle fraction of the soft steroid.

A variety of androstene-derived soft steroids are disclosed in U.S. Pat. No. 5,981,517, which is incorporated herein by reference in its entirety. This discloses a range of soft steroids suitable for use in the present invention, although it will be appreciated that the invention is not limited merely to those compounds listed.

Soft steroids which may be part of the present inventive medicament comprise those defined by the structural formula:

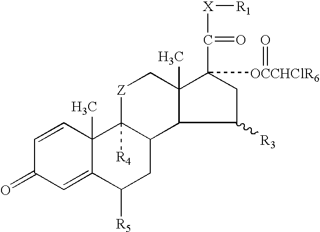

wherein
R₁ is C₁-C₄ alkyl, which is unsubstituted or which bears one substituent selected from the group consisting of chloro, fluoro, C₁-C₄ alkoxy, C₁-C₄ alkylthio, C₁-C₄ alkylsulfinyl and C₁-C₄ alkylsulfonyl;
R₃ is hydrogen, α-hydroxy, α-hydroxy, β-methyl, β-methyl ═CH₂, or α- or

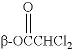

R₄ is hydrogen, fluoro, or chloro;
R₅ is hydrogen, fluoro, chloro or methyl;
R₆ is hydrogen, chloro or methyl;

### X is —O— or —S—;

Z is carbonyl, β-hydroxymethylene or β-chloromethylene;
and the dotted line in ring A indicates that the 1,2-linkage is saturated or unsaturated.

Within this group of compounds, the following subgroups are preferred:

(1) compounds in which R₃ is H, R₄ is H or F and R₅ is H, F or CH₃;
(2) compounds in which R₃ is α-CH₃ or β-CH₃, R₄ is H or F and R₅ is H F or CH₃; and

### (3) R₃ is α-OH, β-OH α-OCOCHCl₂ or β-OCOCHCl₂, R₄ is H or F and R₅ is H, F or CH₃.

Particularly preferred compounds are those having one or more of the following structural characteristics:
(1) R₁ is unsubstituted C₁-C₄ alkyl or chloromethyl, especially when R₁ is unsubstituted alkyl, most especially when R₁ is ethyl or methyl;
(2) X is —O—;
(3) Z is β-hydroxymethylene;
(4) the 1,2-linkage is unsaturated; especially when the R₃, R₄ and R₅ variables are the preferred ones described in the preceding paragraph.

One group of especially preferred compounds is defined by the structural formula:

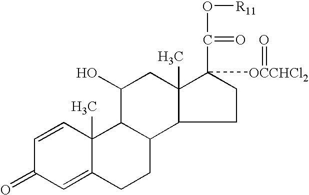

wherein R₁₁ is methyl, ethyl, isopropyl or chloromethyl, especially when R₁₁ is methyl, ethyl or isopropyl.

An exceptionally preferred soft steroid compound is ethyl 17α-dichloroacetoxy-11β-hydroxyandrosta-1,4-dien-3-one-17β-carboxylate, known as etiprednol didoacetate (EDA), which is defined by the structural formula:

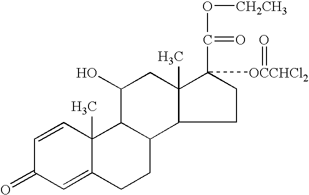

A further exceptionally preferred soft steroid compound, is chloromethyl 17α-ethoxycarbonyloxy-11β-hydroxyandrosta-1,4-diene-3-one-17β-carboxylate, known as loteprednol etabonate (LE), which is defined by the structure formula:

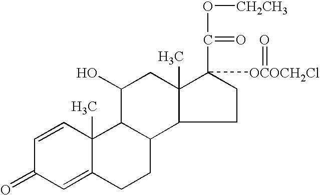

An alternative group of soft steroids which may be part of the present inventive formulation comprise those defined by the structural formula:

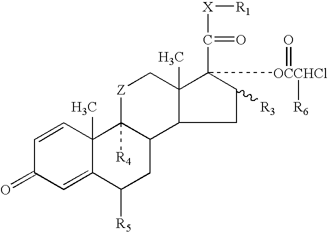

wherein:
R₆ is H or CH3, particularly when R₆ is CH3, and the remaining structural variables are as defined above.

Within this group of compounds, the following subgroups are preferred:
(1) compounds in which R₃ is H, R₄ is H or F and R₅ is H, F or CH₃;
(2) compounds in which R₃ is α-CH₃ or β-CH₃, R₄ is H or F and R₅ is H, F or CH₃; and
(3) R₃ is α-OH, β-OH, α-OCOCHCl₂ or β-OCOCHCl₂, R₄ is H or F and R₅ is H, F or CH₃.

Particularly preferred compounds are those having one or more of the following structural characteristics:
(1) R₁ is unsubstituted C₁-C₄ alkyl or chloromethyl, especially when R₁ is unsubstituted alkyl, most especially when R₁ is ethyl or methyl;
(2) X is —O—;
(3) Z is β-hydroxymethylene;
(4) the 1,2-linkage is unsaturated; especially when the R₃, R₄ and R₅ variables are the preferred ones described in the preceding paragraph. Most especially preferred derivatives are defined by the structural formulas:

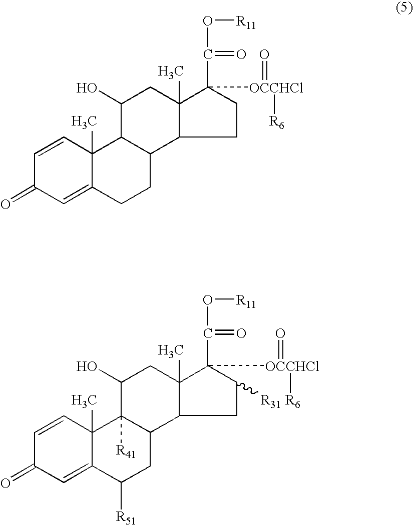

wherein R₁₁ is methyl ethyl, isopropyl or chloromethyl, R₃₁ is α-CH₃ or β-CH₃, R₄₁ is H or F and R₅₁ is H or F, especially when R₁₁ is methyl, ethyl or isopropyl.

Examples of suitable excipients for use in the present invention are monosaccharides, disaccharides and polysaccharides and derivatives thereof, for example lactose, sucrose, glucose, mannitol, xylitol trehalose, although other suitable excipients may be used. A particularly preferred excipient is lactose monohydrate.

One advantage is to provide a medicament containing a soft steroid, preferably etriprenol dicloacetate or lotiprednol etabonate, most preferably etiprednol didoacetate, which is suitable for use in a dry powder inhaler.

Preferably, in each aspect of the present invention the volume mean diameter of the soft steroid particles is less than the diameter of the excipient particles. Preferably, the volume mean diameter of the soft steroid particles is more than about 3 times smaller than the mean diameter of the excipient particles. More preferably, the volume mean diameter of the soft steroid particles is more than about 5 times smaller than the mean diameter of the excipient particles. For example, when the volume mean diameter of the soft steroid particles is less than about 10 μm, the volume mean diameter of the excipient particles is at least about 50 μm, preferably at least about 80 μm.

Preferably, at least about 50% of the soft steroid particles by weight have a diameter less than about 10 μm. More preferably, at least about 50% of the soft steroid particles by weight have a diameter less than about 5 μm. Most preferably, at least about 50% of the soft steroid particles by weight have a diameter less than about 3 μm. For example, when the soft steroid particles have a volume mean diameter of from about 1.5 to 3 at least 90% of those particles by weight will have a mean diameter less than about 3 μm.

Preferably, at least about 30% of the excipient particles by weight have a diameter less than 100 μm, at least about 50% of the excipient particles by weight have a mean diameter less than 500 μm. Preferably, no more than 50% of the excipient particles by weight have a diameter less than about 10 μm. For example, when the excipient particles have a volume mean diameter of from about 60 to 100 μm, about 5 to 15% of those particles by weight have a diameter less than about 10 μm.

A further advantage is to provide a medicament capable of being delivered via dry powder inhalers and having excellent uniformity of dose per delivery.

A still further advantage of the preferred embodiments is to provide improved efficiency in the administration of the medicament, thereby increasing its therapeutic index, via inhalation delivery.

Advantageously, the resulting medicament is of decreased toxicity due to the presence of a soft steroid active compound, as compared with the alternatives based upon conventional steroids, thereby further improving its therapeutic index.

Another advantage is the improved stability of the medicament due to its incorporation in a dry powder formulation.

Another advantage is that the stability of the medicament is improved further via its incorporation in a dry powder inhaler.

Further features and advantages of the presently disclosed formulation and method of preparation thereof will become more readily apparent to those having ordinary skill in the art to which the present disclosure relates to the drawings and detailed description.

A first embodiment of the present invention provides formulations containing soft steroids, for example for use as a medicament to be administered via dry powder inhalers to the lungs to exert either localised or systemic effects, said soft steroids being combined in an homogenous blend with a suitable excipient.

Preparation of a Homogenous Blend of the Soft Steroid Particles and Excipient Particles May be achieved by simply admixing the two particle populations by a technique conventionally known in the art. Mixing of the particles may typically be effected by diffusive, shear or convective mixing, preferably by kinetic mixing.

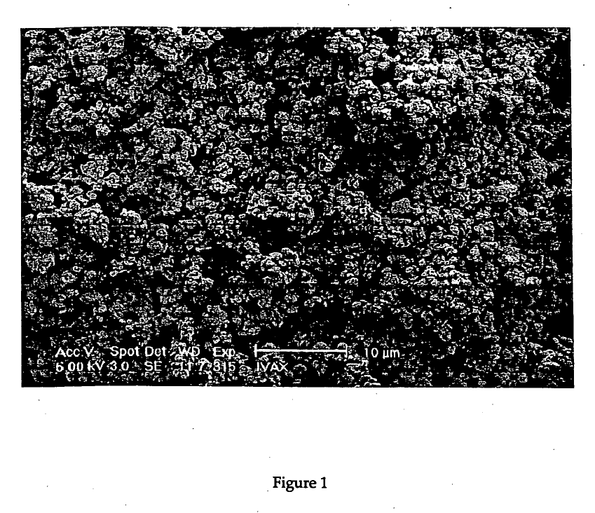

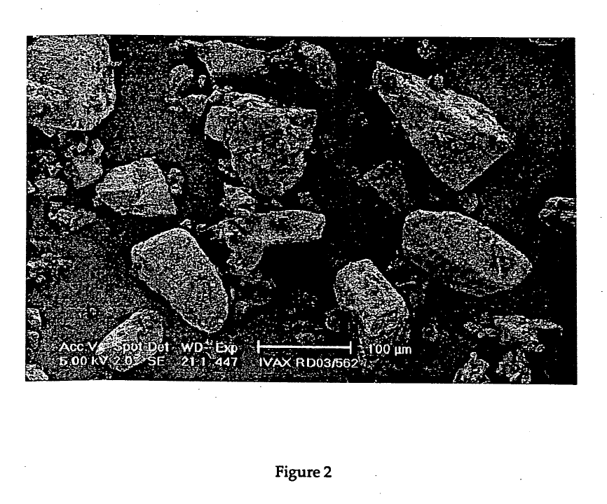

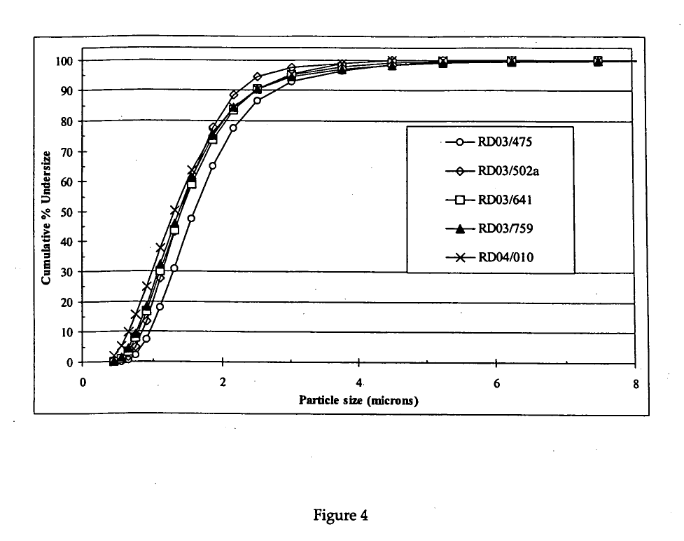

Typically, a homogenous blend is achieved by micronizing, or otherwise reducing the particles size by techniques known in the art, the soft steroid to a known particle size (See e.g. FIGS. 1 and 4), then separately preparing the excipient particles in a conventional manner, to achieve its required particle size (See e.g. FIG. 2). The micronized soft steroid particles are then allowed to pass through a mesh with a size of typically about 250 μm. The two populations of particles are then mixed such that the soft steroid particles are generally distributed amongst the excipient particles. Preferably kinetic mixing techniques of the type known in the art are used.

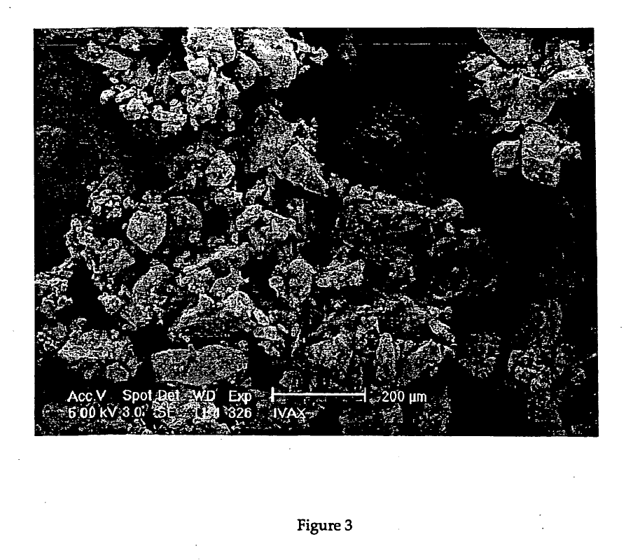

Preferred embodiments of the present invention contain a concentration of soft steroid particles in the range of up to about 50% by wt in relation to the excipient particles, and preferably in the range of about 1 to about 10% and most preferably in the range of about 3 to about 7%. The even distribution of soft steroid and excipient particles can be seen in the scanning electron micrograph of FIG. 3. This may alternatively be assessed by means of measuring soft steroid concentrations in samples taken from various locations of the blend.

Preferred embodiments of the invention should contain soft steroid particles with a volume mean diameter (VMD) of less than about 20 micrometers, preferably contain soft steroid particles with a VMD of less than about 10 micrometers and more preferably contain soft steroid particles with a VMD of less than about 5 micrometers and most preferably, the average VMD of the soft steroid particles is from about 1.5 to 2.5 micrometers Soft steroid particles of the required VMD may be obtained by any of micronisation, crystalliation, condensation from vapour, spray drying, freeze drying and any other suitable technique known in the art.

Further, preferred embodiments of the present invention should include excipient particles with a VMD in the range of about 10 to about 1000 micrometers, and preferably in the range from about 15 to about 250 micrometers and most preferably in the range of about 20 to about 100 micrometers. Excipient particles of the correct VMD may be obtained by any of micronisation, crystalliation, condensation from vapour, spray drying, freeze drying and any other suitable technique known in the art. Additionally, different grades of excipient may be mixed to obtain the correct range of particle size and it may also be possible to grind or mill larger excipient particles and then fractionate by passing the mix through meshes of different size ranges.

The volume mean diameter of the particles can be measured by conventional light scattering techniques, such as by laser diffraction techniques which are well-known in the art, for example via the use of a helium neon laser which, when interrupted with a particle flow, creates a diffraction pattern on a detector. A computer-encoded algorithm can be used to translate this diffraction pattern to particle size data.

In a preferred delivered dose of the preferred formulations of the present invention, 10 to 5000 micrograms of active compound will be delivered and preferably 100 to 500 micrograms, and most preferably about 200 micrograms will be delivered per dose.

Thus, preferred formulations of the present invention enable delivery via dry powder inhalers having excellent uniformity of dose per delivery with a high fine particle fraction of soft steroid.

The dry compositions of the present invention are surprisingly stable, in comparison to compositions comprising soft steroid and aqueous solvents. For example, a 5 wt % EDA/95 wt % lactose dry mix according to the invention (EDA volume mean diameter 1.58 μm/lactose volume mean diameter 80 μm) was found to be stable for over 1 month at 40° C./75% RH. In comparison, solutions prepared by dissolving 2 wt % EDA in a) pure methanol, b) pure ethanol, c) methanol+10 wt % water (pH 4.5) and d) ethanol+10% water (pH 4.5), were stable for 2 days, 2 days, 4 days and 4 days, respectively.

Whilst it is believed that a person of ordinary skill in the art can, by using the preceding description, utilize the present invention to its fullest extent, the invention shall now be further described by way of exemplification, which examples are provided to be merely illustrative and not limiting on the scope of the claims.

### EXAMPLES

### Example 1

### Preparation of Powdered EDA

A population of etiprednol dicloacetate (EDA) particles was prepared for use in the present invention from a coarse material source. The coarse material as supplied was initially micronized to provide a powder wherein the population of particles had an average volume mean diameter of from 1.5 to 1.7 and then sieved through a 250 μm sieve to produce a powder comprising a population of EDA particles having a volume mean diameter of 1.8 μm, as determined by a conventional light scattering dry dispersion method using a Sympatec HELOS BF Magic Particle Size Analyser. The scanning electron micrograph of a typical batch of micronized etiprednol dicloacetate is shown in FIG. 1. The particle size distributions of several batches of micronized etiprednol dicloacetate are shown in FIG. 4.

The powder which passed through the sieve was analysed further and found to comprise 100% of particles having a mean diameter of less than 10 μm, with more than 90% of particles having a mean diameter of less than 3 μm The powder had a true density of 1.3 gcm⁻³, as measured by using a Helium Pycnometer.

### Example 2

### Preparation of Powdered Lactose

A population of α-lactose monohydrate particles was prepared for use in the present invention from a coarse material source. The coarse material was initially crystallized, milled and sieved to provide a powder wherein the population of particles had a mean diameter of about 80 μm with about 10% of the particles having a mean diameter less than 10 μm. The scanning electron micrograph of a typical batch of lactose is shown in FIG. 2. The particle size distributions of several batches of lactose, as measured by laser diffraction are shown in Table 1.

TABLE 1
Particle size distribution distributions of several batches of lactose

| Cumulative % | | | | | |
|---|---|---|---|---|---|
| undersize | Batch 1 | Batch 2 | Batch 3 | Batch 4 | Batch 5 |
| 10 μm | 7 | 10 | 11 | 11 | 11 |
| 30 μm | 17 | 20 | 19 | 19 | 16 |
| 60 μm | 48 | 35 | 36 | 30 | 31 |
| 90 μm | 78 | 55 | 58 | 49 | 58 |
| 174 μm | 100 | 97 | 97 | 96 | 99 |
| 250 μm | 100 | 100 | 100 | 100 | 100 |

### Example 3

### Preparation of EDA/Lactose Blend

A 2.5 kg blend consisting of 4.7 wt % EDA and 95.3 wt % α-lactose monohydrate was prepared by mixing sieved particles formed in Example 1 with a population of lactose particles having a mean particle size of 80 μm and a fine particle concentration (%<10 μm) of 10.5%. The particles were subjected to kinetic mixing in a 61 stainless steel vessel with a ca. 50% headspace using a Turbula T10B for 20 minutes at 32 rpm. The mixture was then passed through a 355 μm sieve to form the homogenous blend of the present invention. FIG. 3 shows the scanning electron micrograph of a typical batch of the blend.

### Example 4

### Pharmaceutical Testing

Three samples of the homogenous blend were filled into three multi-dose dry powder inhalers-Airmax, with a fill weight range of 0.73-0.77 g, thereby to give not less than 200 shots from each inhaler.

The through-life uniformity of delivered dose from each inhaler was tested by discharging 10 single doses, 3 at beginning, 4 at middle and 3 at end of device life, at 4 kPa pressure drop across the device in a 41 volume. The drug that was collected was then recovered and analysed using a HPLC assay.

The aerodynamic particle size distribution of EDA in the blend was measured using a multistage liquid impinger (ELI), using the same conditions as in delivered dose determination. The drug deposited in each stage of the MSLI was then recovered and quantified using HPLC assay.

The pharmaceutical results of 6 batches of the blend are listed in Table 2.

TABLE 2
Mean drug per actuation (DPA), relative standard deviation
(RSD) of DPA and fine particle fraction (FPF) of etiprednol
dicloacetate from the Airmax Multidose Dry Powder
Inhalers containing several batches of the EDA and lactose
blend (target dose-200 μg)

| Blends | DPA (μg) | RSD (%) | FPF (%) |
|---|---|---|---|
| Batch A | 209 | 8 | 51 |
| Batch B | 205 | 8 | 58 |
| Batch C | 191 | 11 | 65 |
| Batch D | 199 | 11 | 59 |
| Batch E | 203 | 9 | 55 |
| Batch F | 192 | 8 | 60 |

The pharmaceutical data from each inhaler indicated that each inhaler containing the blend consistently deliver the target EDA dose per actuation. The relative standard deviation is about 8-11%, which is substantially lower than the RSD (ca. 20%) of other dry powder inhalers, suggesting that the blend leads to precise and accurate dose delivery throughout the inhaler life. Each batch of blend results in fine particle fraction of typically 50%, which is substantially higher than the FPF (ca. 25%) of other dry powder inhalers. Therefore, the compositions are likely to be very suitable for administration of soft steroids for the treatment of asthma.

### Example 5

### Stability Testing

The Airmax Multi-dose Dry Powder Inhalers containing the homogeneous blend were stored unprotected in accelerated conditions, i.e. 40° C./75% relative humidity for one month. The inhalers were then subjected to extensive pharmaceutical testing.

Table 3 shows the pharmaceutical results of a blend before and after storage at the accelerated conditions for a month.

TABLE 3
The stability of a blend after 2 weeks and 1 month storage at
40° C./75% RH.

| Time point | Mean DPA/μg | % RSD | FPF/% |
|---|---|---|---|
| Initial (n = 3) | 191 | 11 | 65 |
| 2 weeks (n = 4) | 187 | 9 | 60 |
| 1 Month (n = 4) | 195 | 8 | 61 |

The results show that the inhalers are as consistent and precise in teams of delivered dose uniformity, and as efficient in terms of fine particle fraction as the inhalers prior to storage. There is no deterioration in the performance of the blend after exposure to the accelerated conditions for a month. Therefore, the compositions are suitably stable.

What is claimed is:

1. A composition comprising particles of at least one soft steroid and at least one excipient, wherein said at least one soft steroid particles have a volume mean diameter of less than about 20 micrometers and said at least one excipient particles have a volume mean diameter in the range of about 10 to about 1000 micrometers.

2. A composition as claimed in claim 1, wherein the volume mean diameter of the soft steroid particles is less than the mean diameter of the excipient particles.

3. A composition as claim 2, wherein the volume mean diameter of the soft steroid particles is more than about 3 times smaller than the mean diameter of the excipient particles.

4. A composition as claimed in claim 3, wherein the volume mean diameter of the soft steroid particles is more than 5 times smaller than the mean diameter of the excipient particles.

5. A composition as claimed in any one of the preceding claims, wherein at least 50 wt % of the soft steroid particles have a diameter less than about 10 μm.

6. A composition as claimed in claim 5, wherein at least 50 wt % of the soft steroid particles have a diameter less than about 5 μm.

7. A composition as claimed in claim 6, wherein at least 50 wt % of the soft steroid particles have a diameter less than about 3 μm.

8. A composition as claimed in claim 8, wherein at least 90 wt % of the soft steroid particles have a diameter of less than about 3 μm.

9. A composition as claimed in any one of the preceding claims, wherein the volume mean diameter of the said soft steroid particles is less than about 10 μm.

10. A composition as claimed in claim 8, wherein said soft steroid particles have an average volume mean diameter of about 5 μm or less.

11. A composition as claimed in claim 9, wherein said soft steroid particles have an average volume mean diameter from about 0.5 to about 5 μm.

12. A composition as claimed in claim 10, wherein said soft steroid particles have an average volume mean diameter from about 1.5 to about 3 μm.

13. A composition as claimed in any one of the preceding claims, wherein said excipient particles have an average volume mean diameter in the range from about 15 to about 250 micrometers.

14. A as claimed in claim 12, wherein said excipient particles have an average volume mean diameter in the range of about 20 to about 100 micrometers.

15. A composition as claimed in any one of the preceding claims, wherein at least about 30 wt % of the excipient particles have a diameter less than 100 μm.

16. A composition as claimed in claim 15, wherein no more than about 50 wt % of the excipient particles have a diameter less than about 10 μm.

17. A composition as claimed in any one of the preceding claims, wherein at least about 50 wt % of the excipient particles have a diameter less than 500 μm.

18. A composition as claimed in any one of the preceding claims, wherein said composition is a dry homogenous blend.

19. A composition as claimed in any one of the preceding claims, wherein said composition is formed by mixing of the soft steroid and excipient particles.

20. A composition as claimed in any one of the preceding claims, wherein the concentration of said soft steroid particles is up to about 50% by wt in relation to the excipient particles.

21. A composition as claimed in claim 20, wherein the concentration of said soft steroid particles is in the range of about 1 to about 10 percent by wt in relation to the excipient particles.

22. A composition as claimed in claim 21, wherein the concentration of said soft steroid particles is in the range of about 3 to about 7 percent by wt in relation to the excipient particles.

23. A composition as claimed in any one of the preceding claims, wherein said soft steroid is defined by the structural formula: 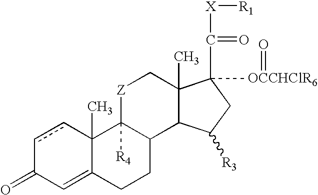
wherein
R₁ is C₁-C₄ alkyl, which is unsubstituted or which bears one substituent selected from the group consisting of chloro, fluoro, C₁-C₄ alkoxy, C₁-C₄ alkylthio, C₁-C₄ alkylsulfinyl and C₁-C₄ alkylsulfonyl;
R₃ is hydrogen, α-hydroxy, β-hydroxy, α-methyl, β-methyl ═CH₂, or α or 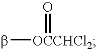
R₄ is hydrogen; fluoro, or chloro;
R₅ is hydrogen, fluoro, chloro or methyl;
R₆ is hydrogen, chloro or methyl;
X is —O— or —S—;
Z is carbonyl, β-hydroxymethylene or β-chloromethylene;
and the dotted line in ring A indicates that the 1,2-linkage is saturated or unsaturated.

24. A composition as claimed in claim 23, wherein R₃ is H, R₄ is H or F and R₅ is H, F or CH₃; or R₃ is α-CH₃ or CH₃, R₄ is H or F and R₅ is H, F or CH₃; or R₃ is α-OH, β-OH, α-OCOCHCl₂ or β-OCOCHCl₂, R₄ is H or F and R₅ is H, F or CH₃.

25. A composition as claimed in claim 23, wherein said soft steroid comprises one or more of the following structural characteristics:
(5) R₁ is unsubstituted C₁-C₄ alkyl or chloromethyl, especially when R₁ is unsubstituted alkyl, most especially when R₁ is ethyl or methyl;
(6) X is —O—;
(7) Z is β-hydroxymethylene;
the 1,2-linkage is unsaturated; and
(4) R₆ is Cl.

26. A composition as claimed in claim 23, wherein said soft steroid is defined by the structural formula: 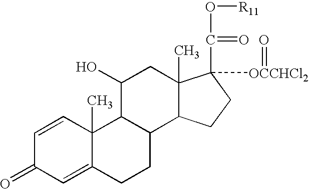
wherein R₁₁ is methyl, ethyl, isopropyl or chloromethyl, especially when R₁₁ is methyl, ethyl or isopropyl.

27. A composition as claimed in any one of the preceding claims, wherein said soft steroid is etiprednol dicloacetate.

28. A composition as claimed in any one of the preceding claims, wherein said soft steroid is loteprednol etabonate.

29. A composition as claimed in claim 23, wherein said soft steroid is defined by the structural formula: 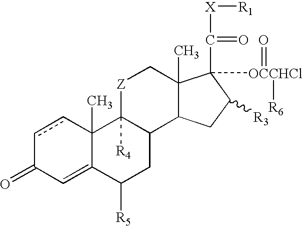
wherein
R₆ is H or CH3, particularly when R₆ is CH3,
R₁ is C₁-C₄ alkyl, which is unsubstituted or which bears one substituent selected from the group consisting of chloro, fluoro, C₁-C₄ alkoxy, C₁-C₄ alkylthio, C₁-C₄ alkylsulfinyl and C₁-C₄ alkylsulfonyl;
R₃ is hydrogen, α-hydroxy, β-hydroxy, α-methyl, β-methyl ═CH₂, or α- or 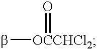
R₄ is hydrogen; fluoro, or chloro;
R₅ is hydrogen; fluoro, chloro or methyl;
X is —O— or —S—;
Z is carbonyl, β-hydroxymethylene or chloromethylene;
and the dotted line in ring A indicates that the 1,2-linkage is saturated or unsaturated.

30. A composition as claimed in claim 23, wherein the soft steroid comprises the following sub-groups
(4) compounds in which R₃ is H, R₄ is H or F and R₅ is H, F or CH₃; or
(5) compounds in which R₃ is α-CH₃ or β-CH₃, R₄ is H or F and R₅ is H, F or CH₃; or
(6) R₃ is α-OH, POOH, α-OCOCHCl₂ or β-OCOCHCl₂, R₄ is H or F and R₅ is H, F or CH₃.

31. A composition as claimed in claim 23, wherein the soft steroid comprises one or more of the following structural characteristics
(6) R₁ is unsubstituted C₁-C₄ alkyl or chloromethyl especially when R₁ is unsubstituted alkyl, most especially when R₁ is ethyl or methyl;
(7) X is —O—;
(8) Z is β-hydroxymethylene;
the 1,2-linkage is unsaturated.

32. A composition as claimed in claim 23, wherein said soft steroid is defined by the structural formula: 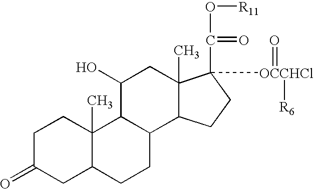
wherein R₁₁ is methyl, ethyl, isopropyl or chloromethyl, and R₆ is H or CH₃, especially when R₁₁ is methyl, ethyl or isopropyl.

33. A composition as claimed in claim 23, wherein said soft steroid is defined by the structural formula: 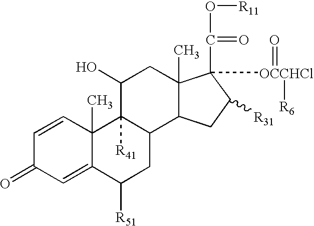
wherein R₁₁ is methyl, ethyl, isopropyl or chloromethyl, R₃₁ is α-CH₃ or β-CH₃, R₄₁ is H or F, R₅₁ is H or F, R₆ is H or CH₃, especially when R₁₁ is methyl, ethyl or isopropyl.

34. A composition as claimed in any one of the preceding claims, wherein said excipient comprises one or more of lactose, sucrose, glucose, mannitol, xylitol, trehalose, or other monosaccharides, disaccharides or polysaccharides, or derivatives thereof.

35. A composition as claimed in claim 34, wherein said excipient is lactose monohydrate.

36. A medicament suitable for use in a dry powder inhaler comprising particles of at least one soft steroid and at least one excipient, wherein said at least one soft steroid particles have a volume mean diameter of less than about 20 micrometers and said at least one excipient particles have a volume mean diameter in the range of about 10 to about 1000 micrometers.

37. A medicament as claimed in claim 36, wherein the volume mean diameter of the soft steroid particles is less than the mean diameter of the excipient particles.

38. A medicament as claim 37, wherein the volume mean diameter of the soft steroid particles is more than about 3 times smaller than the mean diameter of the excipient particles.

39. A medicament as claimed in claim 38, wherein the volume mean diameter of the soft steroid particles is more than 5 times smaller than the mean diameter of the excipient particles.

40. A medicament as claimed in claim 36, wherein at least 50 wt % of the soft steroid particles have a diameter less than about 10 μm.

41. A medicament as claimed in claim 40, wherein at least 50 wt % of the soft steroid particles have a diameter less than about 5 μm.

42. A medicament as claimed in claim 41, wherein at least 50 wt % of the soft steroid particles have a diameter less than about 3 μm.

43. A medicament as claimed in claim 42, wherein at least 90 wt % of the soft steroid particles have a diameter of less than about 3 μm.

44. A medicament as claimed in claim 36, wherein the volume mean diameter of the said soft steroid particles is less than about 10 μm.

45. A medicament as claimed in claim 44, wherein said soft steroid particles have a volume mean diameter of about 5 μm or less.

46. A medicament as claimed in claim 45, wherein said soft steroid particles have a volume mean diameter from about 0.5 to about 5 μm.

47. A medicament as claimed in claim 55, wherein said soft steroid particles have a volume mean diameter from about 1.5 to about 3 μm.

48. A medicament as claimed in claim 36, wherein said excipient particles have a volume mean diameter in the range from about 15 to about 250 micrometers.

49. A medicament as claimed in claim 48, wherein said excipient particles have a volume mean diameter in the range of about 20 to about 100 micrometers.

50. A medicament as claimed in claim 36, wherein at least about 30 wt % of the excipient particles have a diameter less than 100 μm.

51. A medicament as claimed in claim 50, wherein no more than about 50 wt % of the excipient particles have a diameter less than about 10 μm.

52. A medicament as claimed in claim 36, wherein at least about 50 wt % of the excipient particles have a diameter less than 500 μm.

53. A medicament as claimed in claim 36, wherein said medicament comprises or consists of a dry homogenous blend of said soft steroid particles and said excipient particles.

54. A medicament as claimed in claim 53, wherein said medicament is formed by mixing of the soft steroid and excipient particles.

55. A medicament as claimed in claim 36, wherein the concentration of said soft steroid particles is up to about 50% by wt in relation to the excipient particles.

56. A medicament as claimed in claim 55, wherein the concentration of said soft steroid particles is in the range of about 1 to about 10 percent by wt in relation to the excipient particles.

57. A medicament as claimed in claim 56, wherein the concentration of said soft steroid particles is in the range of about 3 to about 7 percent by wt in relation to the excipient particles.

58. A medicament as claimed in claim 36, wherein said soft steroid is defined by the structural formula: 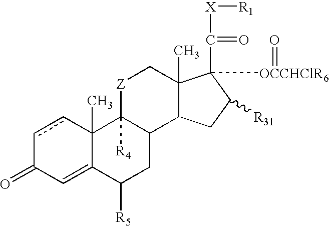
wherein
R₁ is C₁-C₄ alkyl, which is unsubstituted or which bears one substituent selected from the group consisting of chloro, fluoro, C₁-C₄ alkoxy, C₁-C₄ alkylthio, C₁-C₄ alkylsulfinyl and C₁-C₄ alkylsulfonyl;
R₃ is hydrogen, α-hydroxy, β-hydroxy, α-methyl, β-methyl ═CH₂, or a or 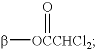
R₄ is hydrogen; fluoro, or chloro;
R₅ is hydrogen, fluoro, chloro or methyl;
R₆ is hydrogen, chloro or methyl;
X is —O— or —S—;
Z is carbonyl, β-hydroxymethylene or β-chloromethylene;
and the dotted line in ring A indicates that the 1,2-linkage is saturated or unsaturated.

59. A medicament as claimed in claim 58, wherein
R₃ is H, R₄ is H or F and R₅ is H, F or CH₃; or
R₃ is α-CH₃ or CH₃, R₄ is H or F and R₅ is H, F or CH₃; or
R₃ is α-OH, β-OH, α-OCOCHCl₂ or β-OCOCHCl₂, R₄ is H or F and R₅ is H, F or CH₃.

60. A medicament as claimed in claim 59, wherein said soft steroid comprises one or more of the following structural characteristics:
R₁ is unsubstituted C₁-C₄ alkyl or chloromethyl, especially when R₁ is unsubstituted alkyl, most especially when R₁ is ethyl or methyl;
X is —O—;
Z is β-hydroxymethylene;
the 1,2-linkage is unsaturated; and
R₆ is Cl.

61. A medicament as claimed in claim 58, wherein said soft steroid is defined by the structural formula: 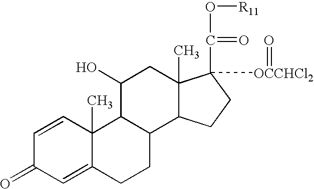
wherein R₁₁ is methyl, ethyl, isopropyl or chloromethyl, especially when R₁₁ is methyl, ethyl or isopropyl.

62. A medicament as claimed in claim 36, wherein said soft steroid is etiprednol dicloacetate.

63. A medicament as claimed in claim 36, wherein said soft steroid is loteprednol etabonate.

64. A medicament as claimed in claim 58, wherein said soft steroid is defined by the structural formula: 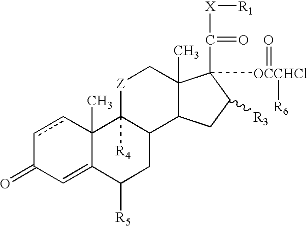
wherein
R₆ is H or CH₃, particularly when R₆ is CH₃,
R₁ is C₁-C₄ alkyl, which is unsubstituted or which bears one substituent selected from the group consisting of chloro, fluoro, C₁-C₄ alkoxy, C₁-C₄ alkylthio, C₁-C₄ alkylsulfinyl and C₁-C₄ alkylsulfonyl;
R₃ is hydrogen, α-hydroxy, β-hydroxy, α-methyl, β-methyl ═CH₂, or α or 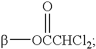
R₄ is hydrogen; fluoro, or chloro;
R₅ is hydrogen; fluoro, chloro or methyl,
X is —O— or —S—;
Z is carbonyl, β-hydroxymethylene or β-chloromethylene;
and the dotted line in ring A indicates that the 1,2-linkage is saturated or unsaturated.

65. A medicament as claimed in claim 58, wherein the soft steroid comprises the following sub-groups compounds in which R₃ is H, R₄ is H or F and R₅ is H, F or CH₃; or
compounds in which R₃ is α-CH₃ or β-CH₃, R₄ is H or F and R₅ is H, F or CH₃; or
R₃ is α-OH, β-OH, α-OCOCHCl₂ or β-OCOCHCl₂, R₄ is H or F and R₅ is H, F or CH₃.

66. A medicament as claimed in claim 58, wherein the soft steroid comprises one or more of the following structural characteristics R₁ is unsubstituted C₁-C₄ alkyl or chloromethyl, especially when R₁ is unsubstituted alkyl, most especially when R₁ is ethyl or methyl;
X is —O—;
Z is β-hydroxymethylene;
the 1,2-linkage is unsaturated.

67. A medicament as claimed in claim 58, wherein said soft steroid is defined by the structural formula: 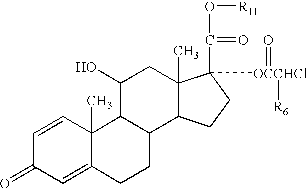
wherein R₁₁ is methyl, ethyl, isopropyl or chloromethyl, and R₆ is H or CH₃, especially when R₁₁ is methyl, ethyl or isopropyl.

68. A medicament as claimed in claim 58, wherein said soft steroid is defined by the structural formula: 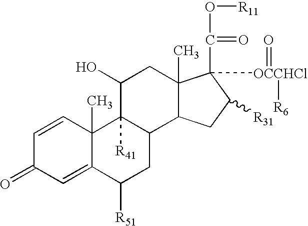
wherein R₁₁ is methyl, ethyl, isopropyl or chloromethyl, R₃₁ is α-CH₃ or β-CH₃, R₄₁ is H or F, R₅₁ is H or F, R₆ is H or CH₃, especially when R₁₁ is methyl, ethyl or isopropyl.

69. A medicament as claimed in claim 36, wherein said excipient comprises one or more of lactose, sucrose, glucose, mannitol, xylitol, trehalose, or other monosaccharides, disaccharides or polysaccharides, or derivatives thereof.

70. A medicament as claimed in claim 36, wherein said excipient is lactose monohydrate.

71. A medicament comprising the composition of claim 1.

72. A medicament suitable for use in a dry powder inhaler comprising the composition as claimed in claim 1.

73. A method of preparing a medicament as claimed in claim 36, wherein said method comprises admixing particles of said soft steroid and said excipient under shear or kinetic mixing conditions.

74. A method of treating a mammal with a medicament administered by way of a dry powder inhaler, said medicament comprising particles of at least one soft steroid and at least one excipient, wherein said at least one soft steroid particles have a volume mean diameter of less than about 20 micrometers and said at least one excipient particles have a volume mean diameter in the range of about 10 to about 1000 micrometers.

75. The method according to claim 74, wherein the medicament is administered for the treatment of respiratory tract or lung based diseases or disorders in mammals, such as asthma.

76. The method according to claim 74, wherein the medicament delivers a dose of about 10 to about 5000 micrograms of soft steroid to said mammal.

77. The method according to claim 74, wherein the medicament delivers a dose of about 100 to about 500 micrograms of soft steroid to said mammal.

78. The method according to claim 74, wherein the medicament delivers a a dose of about 200 micrograms of soft steroid to said mammal.

79. The use of a composition comprising particles of at least one soft steroid and at least one excipient wherein said at least one soft steroid particles have a volume mean diameter of less than about 20 micrometers and said at least one excipient particles have a volume mean diameter in the range of about 10 to about 1000 micrometers in the preparation of a medicament for the treatment of respiratory tract or lung diseases or disorders in a mammal.

80. The use as claimed in claim 79, wherein the medicament is for the treatment of asthma.

81. A medicament according to claim 36 in which the presence of said excipient improves the stability of the soft steroid.
